(19) United States
(12) Patent Application Publication
(10) Pub. No.: US 2018/0350080 A1
(43) Pub. Date: Dec. 6, 2018

(54) Analysis method and system of digital subtraction angiographic images

(71) Applicants: NATIONAL YANG-MING UNIVERSITY, Taipei (TW); Taipei Veterans General Hospital, Taipei (TW)

(72) Inventors: Yi-Hsuan KAO, Taipei (TW); Chung-Jung LIN, Taipei (TW); Jia-Sheng HONG, Taipei (TW)

(21) Appl. No.: 16/001,623

(22) Filed: Jun. 6, 2018

Related U.S. Application Data
(60) Provisional application No. 62/515,674, filed on Jun. 6, 2017.

(51) Int. Cl. G06T 7/136; A61B 6/00; G06T 7/00

(52) U.S. Cl. CPC G06T 7/136; A61B 6/481; A61B 6/4021; A61B 6/527; A61B 6/504; G06T 2207/20224; A61B 6/5205; G06T 7/0012; G06T 2207/10116; G06T 2207/30101; A61B 6/463

(57) ABSTRACT

The present invention discloses an analysis method and system for detecting vascular structures at arterial, capillary, and venous phases from time-series digital subtraction angiographic images. The method comprises the steps of: acquiring a time-series dataset of a subject using at least one rotating x-ray source and detector pair; administrating a contrast media to a blood vessel of the subject during the data acquisition; applying a motion artifact correction to the time-series dataset; and applying a segmentation method to the time-series dataset for identifying vascular structures with different flow patterns of the contrast media. The invention offers the potential to efficiently provide quantitative flow changes inside the clinical operation room without manual selection and motion artifact error.

### BACKGROUND OF THE INVENTION

### 1. Field of the Invention

The present invention relates to a method and system of digital subtraction angiographic images, and more particularly to analysis method and system for detecting vascular structures at arterial, capillary, and venous phases from time-series digital subtraction angiographic images.
Abbreviations:
DSA: digital subtraction angiography
TDC: time-density curve
TTP: time to peak
AUC: area under curve
SIFT: scale-invariant feature transform
ICA: independent component analysis

### 2. Background

Balloon dilatation or install the stent of minimally invasive surgery is needed for brain neck stenosis or obstruction. Currently clinically relevant diagnostic techniques are computed tomography angiography images, magnetic resonance image, and X-ray digital subtraction angiographic (DSA) images.

X-ray DSA images, with its sub-millimeter and sub-second resolutions, is the gold standard for diagnosing cerebrovascular diseases. In this technique, a pair or two pairs of X-ray tube and flat-panel detector are used to acquire projection images of a patient's head at different time point. During the imaging procedure, a bolus of contrast media is injected into a blood vessel. The passage of the contrast media through the brain is recorded on the projected X-ray images. By using a subtraction of the later images with the early baseline images, the artery, capillary, and vein can be illustrated on the subtracted images.

The time-density curve (TDC) measured from a region of interest (ROI) represents the changes in intensity of the contrast bolus passing through the region. It is affected by the bolus characteristics and pathologic conditions (e.g. arterial stenosis or arterio-venous shunts). The advantages of using TDC are that it is less computer-intensive and it provides almost immediate results. For each TDC, seven hemodynamic parameters can be calculated for diagnosis purpose. These parameters are: time to peak (TTP), area under curve (AUC), maximum enhancement, full width half maximum (FWHM), bolus arrival time, maximum wash-in slope, and minimum wash-out slope, as described in paper titled as “Peritherapeutic hemodynamic changes of carotid stenting evaluated with quantitative DSA in patients with carotid stenosis.” published by Teng et al. in American Journal of Neuroradiology 2016; 37:1883-1888.

U.S. Pat. No. 8,929,632 issued to Horz et al. entitled as “Temporal difference encoding for angiographic image sequences” disclosed a method of visualizing changes in blood flow in a DSA image sequence. A time-contrast curve is generated for all pixels in the DSA image sequence. A reference parameter for each time-contrast curve to be used as a first time point is specified. The value of the reference parameter for each time-contrast curve is determined and an arbitrary parameter is specified for each time-contrast curve to be used as a second time point. An output image is generated by applying a color-coding of the difference between the first time point and the second time point for all pixels.

The scan time for each image dataset is about 10 seconds. Patients head motion may cause imperfect subtraction of stationary structures, causing reduced image quality and affecting the analysis results. This motion artifact can cause erroneous measurement of TDC.

Moreover, the cerebral circulation time is defined as the difference between the TTPs of the internal carotid artery and the parietal vein as described in a paper titled as “Monitoring peri-therapeutic cerebral circulation time: a feasibility study using color-coded quantitative DSA in patients with steno-occlusive arterial disease.” published by Lin C J et al. in American Journal of Neuroradiology 2012; 33:1685-1690. It is a quantitative index for evaluating intravascular flow in different vascular disorders such as carotid stenosis, carotid cavernous fistula and peri-therapeutic assessment. However, manual selection for the internal carotid artery and parietal vein is still required and thus measurements are susceptible to intra-observer and inter-observer variations. To establish a more objective measurement, it is needed to avoid manual selection of arterial and venous regions.

Therefore, it is necessary to propose a blood vessel image analysis system and method that can solve the above problems to meet the needs of clinical medicine.

### BRIEF SUMMARY OF THE INVENTION

It is one objective of the present invention to develop an analysis method to detect vascular structures at arterial, capillary, and venous phases from time-series DSA images with erroneous motion artifact.

It is another objective of the present invention to provide an analysis system to detect vascular structures at arterial, capillary, and venous phases from time-series DSA images with erroneous motion artifact.

To achieve the first objective, the present invention provides an analysis method for detecting vascular structures at arterial, capillary, and venous phases from time-series digital subtraction angiographic images, comprising the steps of: (a) acquiring a time-series dataset of a subject using at least one rotating x-ray source and detector pair; (b) administrating a contrast media to a blood vessel of the subject during the data acquisition in step (a); (c) applying a motion artifact correction to the time-series dataset; and (d) applying a segmentation method to the time-series dataset for identifying vascular structures with different flow patterns of the contrast media.

According to one feature of the present invention, the step (d) further comprises the steps of: (d-1) segmenting the time-series dataset into a plurality of blood vessel images; (d-2) differentiating a plurality of blood vessel images to form a plurality of mask images; and (d-3) measuring the TDC of the corresponding mask images.

To achieve the other objective, the present invention provides an analysis system of digital subtraction angiographic image, receiving time-series dataset of a subject using at least one rotating x-ray source and detector pair, comprising: an data storage unit, used to storage the time-series dataset of the subject; an image preprocessing unit, used to perform a motion artifact correction on the time-series dataset; an image segmentation unit, used to segment the time-series dataset into a plurality of blood vessel images; a mask generation unit, used to differentiate the a plurality of blood vessel images to form a plurality of mask images; a data processing unit, used to identify TDCs of the mask images; and a medical image interface, used to display the TDC, mask images and the blood vessel images

According to one feature of the present invention, the image segmentation unit segments the time-series dataset by using the segmentation method includes but not limited to a clustering technique, a blind source separation technique, or a machine learning technique.

According to one feature of the present invention, the image segmentation unit segments the time-series dataset by using an independent component (ICA) method.

The present invention proposes an analysis method and system of blood vessel image, using a Graphic-User Interface which is a computer programming software to reduce errors of the motion artifacts of digital subtraction angiographic images, to obtain region of interest in real time, to obtain arteries, capillaries and venous images, and to measure the TDC of these three types of blood vessels, and thus can improve the accuracy of the analysis of the severity of each lesion. The segmentation method offers the potential to efficiently provide quantitative flow changes inside the clinical operation room without manual selection, particularly, it can reduce the time the physicians needed to come back and forth between the operating table and the computer.

The analysis method and system according to the present invention have the following advantages:
1. The invention uses correction for motion artifacts.
2. The invention can rapidly calculate, analyze, measure and display the morphology of the ROI in the image.
3. The invention can and quickly provide the arterial, capillaries and venous images and the corresponding TDC. The physicians do not have to manually select the region of interest.
4. The invention allows the physician to immediately assess the severity of the disease and to improve the surgery. It can reduce the time for clinical interpretation and thus is more convenient.

### BREIF DESCRIPTION OF THE DRAWINGS

All the objects, advantages, and novel features of the invention will become more apparent from the following detailed descriptions when taken in conjunction with the accompanying drawings.

FIG. 1 is a flow chart of an analysis method of digital subtraction angiographic images of the present invention.

FIG. 2 is a segmentation flow chart of step (d) of the analysis method of the digital subtraction angiographic image of the present invention.

FIG. 3 is a block diagram of an analysis system of the digital subtraction angiographic image of the present invention.

FIG. 4 is digital subtraction angiographic image before (a) and after (b) alignment correction of motion artifact. (a) the arrows indicate the motion artifact before the correction; (b) the arrows point at the same position, and there is no motion artifact after the correction.

FIG. 5 is scatter plot of TTP (horizontal axis) and AUC (vertical axis) for a dataset of DSA images.

FIG. 6 is three different-phase images obtained by using the ICA method. (a) arterial phase, (b) capillaries phase and (c) venous phase.

FIG. 7 is three mask images obtained by using the Otsu binarization method, (a) arterial phase, (b) capillaries phase and (c) venous phase.

FIG. 8, which applies mask images to a whole set of digital angiographic image series to generate the TDC of (a) arterial phase, (b) capillaries phase and (c) venous phase.

### DETAILED DESCRIPTION OF THE INVENTION

Although the invention has been explained in relation to several preferred embodiments, the accompanying drawings and the following detailed descriptions are the preferred embodiment of the present invention. It is to be understood that the following disclosed descriptions will be examples of present invention, and will not limit the present invention into the drawings and the special embodiments.

To facilitate the above description of the central idea expressed in the column of the invention, the digital subtraction angiographic image is expressed as a specific embodiment. But the scope of implementation is not limited to the following examples. Before disclosing the embodiments of the present invention, the terms in the specification of the present invention are defined. The term “interface” means a display that is displayed on an intelligent device (such as a variety of computers) for operator viewing, operating and inputting instructions. A so-called “unit” means an assembly of a program or programs producing the intended results by the processors of the smart devices described above. The so-called “system” refers to the hardware and software assembly that contains both the smart devices mentioned above and the “unit” mentioned above can produce a final result when connected to operate. The so-called “operators” refers to those who have medical expertise and medical image interpretation ability.

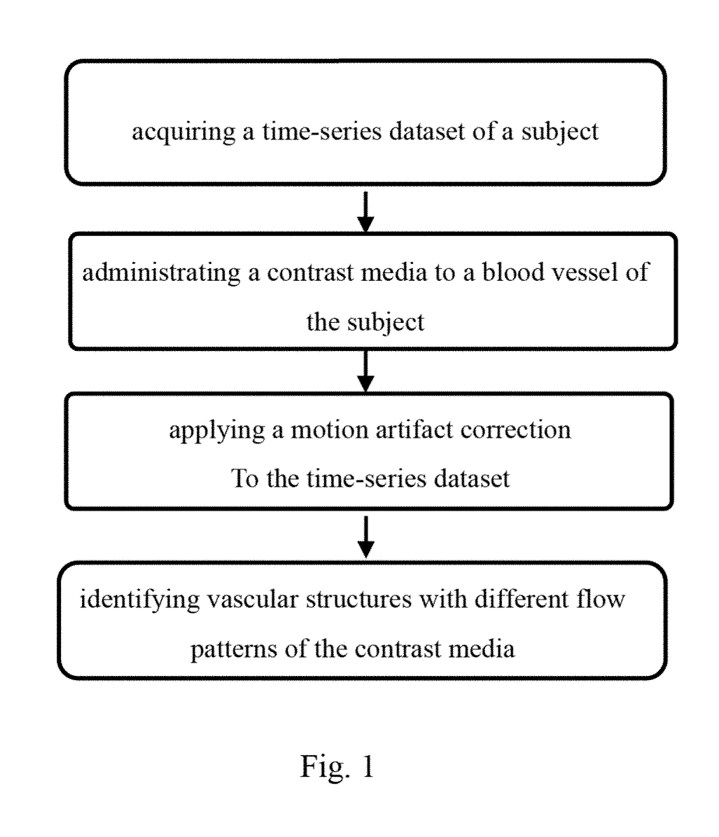

Please refer to FIG. 1, it is a flow chart of an analysis method of digital subtraction angiographic images of the present invention. An analysis method for detecting vascular structures at arterial, capillary, and venous phases from time-series digital subtraction angiographic images, comprising the steps of:
(a) acquiring a time-series dataset of a subject using at least one rotating x-ray source and detector pair;
(b) administrating a contrast media to a blood vessel of the subject during the data acquisition in step (a);
(c) applying a motion artifact correction to the time-series dataset; and
(d) applying a segmentation method to the time-series dataset for identifying vascular structures with different flow patterns of the contrast media.

The contrast media can exhibit the flow of the blood at different times. In the step (a), the time-series dataset is two-dimensional images acquired at an anterior position of the blood vessel, a posterior position of blood vessel, or a lateral positions of the blood vessel. In this invention, since the time-series dataset is acquired by at least one rotating x-ray source and detector pair, the time-series dataset is preferably the digital subtraction angiographic images. And time-series dataset is acquired over a period of time, and the time interval of each image is about 150 milliseconds (ms). The contrast media is injected as a bolus using an injector.

Step (c) further comprises the step of applying a motion artifact correction to the time-series dataset. Preferably, the motion artifact correction is performed by a scale-invariant feature transform (SIFT) process, as described in a paper titled as “SIFT flow: Dense correspondence across scenes and its applications.” published by Liu C, et al. in IEEE Transactions on Pattern Analysis and Machine Intelligence 2011, 33(5):978-994. After performing the registration process as the motion artifact correction, the intensity of the X-ray projection reference image is subtracted from the time-series dataset images to generate DSA images.

In the step (d), the segmentation method includes but not limited to a clustering technique, a blind source separation technique, or a machine learning technique. Applying a segmentation method to the time-series dataset or calculated parametric dataset is used to identify vascular structures at arterial, capillary, and venous phases, and the corresponding time-intensity curves of these three vascular structures.

The cluster technique is a sub-division of statistics methods, and each object is grouped by the fact that grouped objects that have been merged together have the same features but differently grouped objects have significant differences. The blind source separation technique refers to the analysis of the source signals from observed mixed signals. In the machine learning technique, training data set are used to develop supervised or unsupervised learning algorithms for identifying different patterns in the data.

Preferably, in step (d), the segmentation method is an ICA method. ICA method is also a condition of blind source separation. ICA method is a linear transfoiniation method using statistical principle to separate the mixed dataset (the time-series dataset or calculated parametric dataset) into a linear combination of statistically independent non-Gaussian signal sources, and that is, a plurality of independent components of the blood vessel image, such as arteries, capillaries and venous blood flow images and signals.

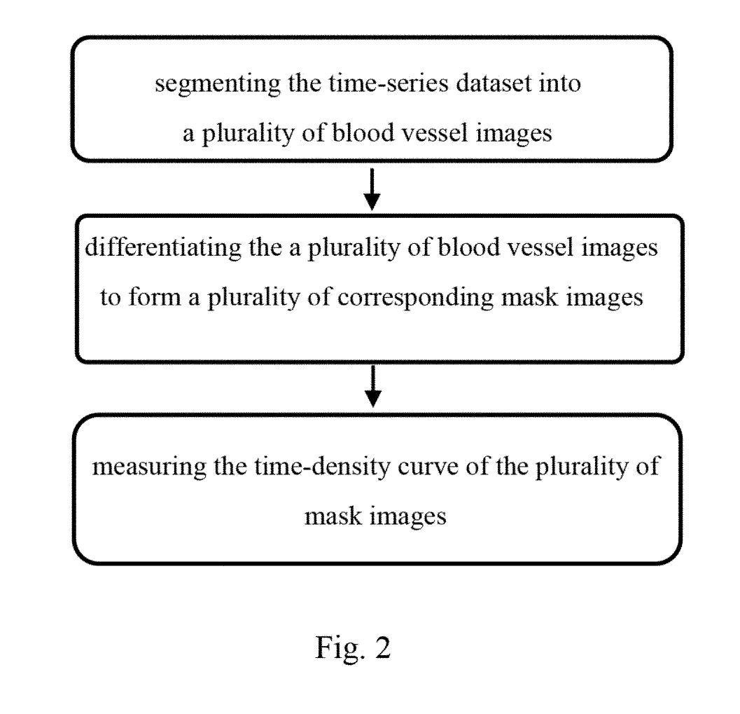

In the step (d), the segmentation method is performed using an original time-series dataset, a subtracted time-series dataset, or a calculated parametric dataset. The subtracted time-series dataset are the images acquired with the contrast media flowing in the blood vessels subtracted by the images acquired before the arrival of the contrast media, and a stationary structure is cancelled out, wherein the stationary structure is bone, gray matter or white matter. The calculated parametric dataset includes but not limited to the time-to-peak image, the maximum-enhancement image, the area-under-curve image. Please refer to FIG. 2, it is a segmentation flow chart of step (d) of the analysis method of the digital subtraction angiographic image of the present invention. The step (d) further comprises the steps of:
(d-1) segmenting the time-series dataset or calculated parametric dataset into a plurality of blood vessel images;
(d-2) differentiating a plurality of blood vessel images to form a plurality of mask images; and
(d-3) measuring the TDC of the mask images.

In the step (d-2), differentiating the plurality of blood vessel images segmented using ICA is obtained by an Otsu binarization method, also named as a thresholding method.

The Otsu binarization method binarizes the blood vessel image based on the same independent component, namely, a gray-scale independent component of the blood vessel image is degenerated into a binary image. The algorithm assumes that the independent component of the blood vessel images include two types of pixels based on the dual-mode histogram (foreground and background pixels), so the calculation of the best threshold value can be done to separate the two types of pixels, making their between-class variance maximal.

According to the steps disclosed above, the method of the present invention avoids the problem of quality differences caused by motion artifacts, and selects region of interest, and identifies the blood flow information in a plurality of blood vessels.

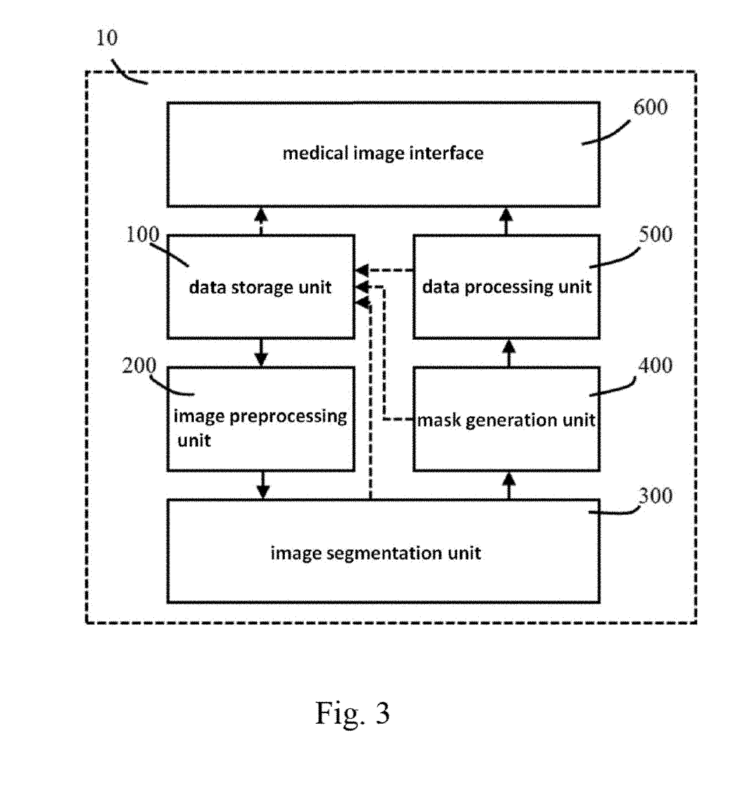

Please refer to FIG. 3, it is a block diagram of an analysis system of the digital subtraction angiographic image of the present invention. The analysis system of digital subtraction angiographic image 10 comprises a data storage unit 100; an image preprocessing unit 200; an image segmentation unit 300; a mask generation unit 400; a data processing unit 500; and a medical image interface 600.

The analysis system of digital subtraction angiographic image receives time-series dataset time-series dataset of a subject using at least one rotating x-ray source and detector pair.

The data storage unit 100 is used to storage the time-series dataset of the subject. The image preprocessing unit 200 is used to perform a motion artifact correction on the time-series dataset. The image segmentation unit 300 is used to segment the time-series dataset into a plurality of blood vessel images using ICA. The mask generation unit 400 is used to differentiate the plurality of blood vessel images segmented using ICA to form a plurality of mask images. The data processing unit 500 is used to identify the TDC of the mask images. The medical image interface 600 is used to display the TDC, mask images, and the plurality of blood vessel images segmented using ICA in the time-series dataset.

The time-series dataset are two-dimensional images acquired at an anterior position of the blood vessel, a posterior position of blood vessel, or a lateral position of the blood vessel.

The image preprocessing flow of the image preprocessing unit 200 includes geometric transformations, color processing, image composite, image denoising, edge detection, image editing, image matching, image enhancement, image digital watermark, image compression, and parametric image calculation. Preferably, the image preprocessing unit 200 uses a scale-invariant feature transform (SIFT) process to perform the motion artifact correction.

The image segmentation unit 300 segments the time-series dataset into a plurality of blood vessel images. The image segmentation unit 300 segments the time-series dataset by using the segmentation method includes but not limited to a clustering technique, a blind source separation technique, a machine learning technique. Preferably, the image segmentation unit 300 segments the time-series dataset by using an ICA method.

The time-series dataset processed by the image segmentation unit 300 are an original time-series dataset, a subtracted time-series dataset, or a calculated parametric dataset. The data processing unit identifies vascular structures at arterial, capillary, and venous phases, and the corresponding TDC of these three vascular structures.

The subtracted time-series dataset are the images acquired with the contrast media flowing in the blood vessels subtracted by the images acquired before the arrival of the contrast media, and the stationary structure is cancelled out, wherein the stationary structure is bone, gray matter or white matter. The calculated parametric dataset includes but not limited to the time-to-peak image, the maximum-enhancement image, the area-under-curve image. The mask generation unit 400 uses an intensity thresholding technique to form a plurality of mask images. Preferably, the mask generation unit 400 uses an Otsu binarization method to differentiate the plurality of blood vessel images segmented using ICA to form a plurality of mask images.

The data processing unit 500 identifies the mask images representing vascular structures at arterial, capillary, and venous phases, and the corresponding TDC of these three vascular structures.

### Embodiment

In step (a) of images acquisition: The following is a typical imaging protocol. Time-series dataset of the X-ray projected images are acquired on a clinical scanner with a frame rate of 6 frames/s for 9˜12 seconds. The image size is 1440×1440 pixels, the field of view is 22 cm, and the pixel size is 0.154×0.154 mm².

In step (b): A power injector is used to inject the contrast media as bolus in the common carotid artery at the C4 vertebral body level. The injection is synchronized with the start of the image acquisition.

In step (a), the scan time for each image dataset is about 10 seconds. Patients head motion may cause imperfect subtraction of stationary structures. This motion artifact can cause erroneous measurement of TDC. It is necessary to perform image registration as the motion artifact correction for the dataset with motion artifact. There are many image registration techniques and they can be characterized to intensity-based and feature based. In the intensity-based techniques, a target image is aligned to a reference using the intensity on the two images. For the feature-based techniques, similar structures on the target and reference images are compared for alignment.

In an embodiment, the scale-invariant feature transform (SIFT) technique is used to register the dynamic time-series dataset to reduce motion artifact. In this technique, a target image is registered to a reference image by comparing local intensity gradients of a predefined region surrounding each pixel on the two images. For each pixel, its 16×16 neighbor pixels are divided into a 4×4 cell array. The orientations of local intensity gradients in a cell are coded into a SIFT descriptor. A SIFT image is composed of SIFT descriptors of all pixels. An objective function similar to that of optical flow is designed to estimate SIFT flow between two SIFT images. On a pixel-by-pixel basis, an optimization process is performed to register a pixel on a target image to a pixel on the reference image. A coarse-to-fine matching scheme is used to accelerate the matching process. After performing the registration process, the intensity of the X-ray projection reference image is subtracted from the target images to generate DSA images.

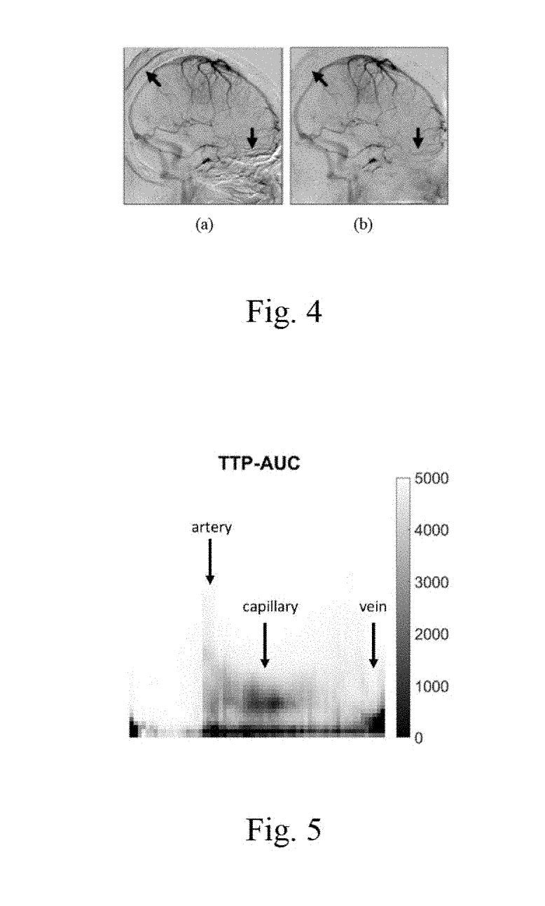

FIG. 4 demonstrates the DSA images before (a) and after (b) the registration process. The arrows in (a) indicate image artifacts caused by motion. The arrows in (b) illustrate these artifacts are successfully removed by using the SIFT registration process.

In step (d), the DSA images is segmented into arterial, capillary, and venous phase. There are many segmentation techniques can be used to achieve this goal. In this invention, a head mask can be produced by applying a threshold to the un-subtracted X-ray projection images. A two-dimensional scatter plot for all pixels inside the head mask can be generated using the TTP and AUC of all pixels as shown in FIG. 5. On FIG. 5, the arterial pixels have small TTP and large AUC, capillary pixels have median TTP and small AUC, and venous pixels have long TTP and large AUC. A clustering technique can be applied to this scatter plots to group pixels into artery, capillary, and vein.

In the above embodiment, a two-dimensional scatter plot is generated by using the TTP and AUC parameters. However, the scatter plot can be extended to multi-dimensions and other parameters also can be used. For example, a seven-dimensional scatter plot can be generated using the TTP, AUC, maximum enhancement, full width half maximum, bolus arrival time, maximum wash-in slope, and minimum wash-out slope parameters. Even higher dimensional scatter plot composed of the dynamic time-series dataset, either before or after subtraction, can be used.

Furthermore, in this invention, there are many segmentation techniques can be used to segment the DSA images into arterial, capillary, and venous phase. For example, the clustering techniques such as: k-means clustering, Fuzzy c-means clustering, Gaussian mixture models can be used. The classification techniques such as Bayesian classification, neural network, machine learning technique also can be used.

Another important segmentation technique is the blind source separation, such as principle component analysis and independent component analysis. In the following sections, we will demonstrate the segmentation of DSA images using the ICA technique. However, this invention is not limited to this technique.

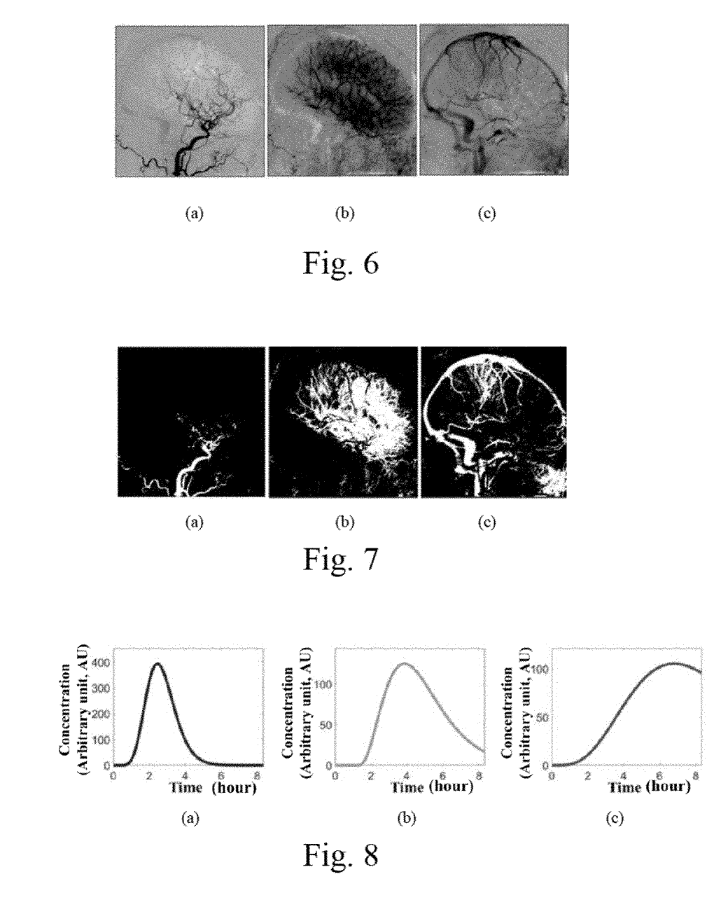

In an embodiment, the FastICA technique, as described in a paper titled as “A fast fixed-point algorithm for independent component analysis.” published by Hyvarinen A. et al. in Neural Comput 1997; 9:1483-1492, is used. The number of output ICA images is set to three. The output ICA images are assumed to be the independent sources which corresponds to arterial, capillary, and venous vessels on the DSA images. FIG. 6 is three-weighted phase image obtained by using the ICA method. (a) arterial phase, (b) capillaries phase and (c) venous phase.

The corresponding TDC are normalized to zero mean and unit variance during the FastICA optimization process. Because the TDC of output ICA images are normalized, we need to generate masks of artery, capillary, and vein for measuring the real TDC. The Otsu binarization method is a thresholding method, as described in a paper titled as “A threshold selection method from gray-level Histograms.” published by Otsu N. in IEEE Transactions on Systems, Man, and Cybernetics 1979, 9(1): 62-66. The Otsu's thresholding method is applied to the output ICA images to generate binary mask images correspond to artery, capillary, and vein. In Otsu's technique, the threshold is determined by a between-class variance maximization algorithm. However, there are pixels assigned to more than one vessel types on the mask images. To remove this ambiguity, the priority for pixel assignment are set as: artery, vein, and capillary. After these re-assignment, binary masks of these three vessel types are used to measure the TDC from the DSA images. FIG. 7 is three mask images obtained by using the Otsu binarization method and they are: (a) arterial phase, (b) capillaries phase and (c) venous phase.

FIG. 8 shows TDC measured from these three masks and they are (a) arterial phase, (b) capillaries phase and (c) venous phase. The seven hemodynamic parameters: TTP, AUC, maximum enhancement, full width half maximum, bolus arrival time, maximum wash-in slope, and minimum wash-out slope, can be measured from these TDC in this invention.

What is claimed is:

1. An analysis method for detecting vascular structures at arterial, capillary, and venous phases from time-series digital subtraction angiographic images, comprising the steps of:
(a) acquiring a time-series dataset of a subject using at least one rotating x-ray source and detector pair;
(b) administrating a contrast media to a blood vessel of the subject during the data acquisition in step (a);
(c) applying a motion artifact correction to the time-series dataset; and
(d) applying a segmentation method to the time-series dataset for identifying vascular structures with different flow patterns of the contrast media.

2. The analysis method as claimed in claim 1, wherein in the step (a), the time-series dataset is two-dimensional images acquired at an anterior position of the blood vessel, a posterior position of blood vessel, or a lateral positions of the blood vessel.

3. The analysis method as claimed in claim 1, wherein in the step (b), the contrast media is injected as a bolus using an injector.

4. The analysis method as claimed in claim 1, wherein in the step (c), the motion artifact correction is performed by a scale-invariant feature transform process.

5. The analysis method as claimed in claim 1, wherein in the step (d), the segmentation method is performed using an original time-series dataset, a subtracted time-series dataset, or a calculated parametric dataset.

6. The analysis method as claimed in claim 1, wherein in the step (d), the segmentation method includes a clustering technique, a blind source separation technique, or a machine learning technique.

7. The analysis method as claimed in claim 1, wherein in the step (d), applying a segmentation method to the time-series dataset for identifying vascular structures at arterial, capillary, and venous phases, and the corresponding time-intensity curves of these three vascular structures.

8. The analysis method as claimed in claim 5, wherein the subtracted time-series dataset are the images acquired with the contrast media flowing in the blood vessels subtracted by the images acquired before the arrival of the contrast media, and stationary structure is cancelled out; wherein the stationary structure is bone, gray matter or white matter.

9. The analysis method as claimed in claim 5, wherein the calculated parametric dataset includes time-to-peak images, maximum-enhancement images, or area-under-curve images.

10. The analysis method as claimed in claim 1, wherein the step (d) further comprises the steps of:
(d-1) segmenting the time-series dataset into a plurality of blood vessel images;
(d-2) differentiating the plurality of blood vessel images to form a plurality of mask images; and
(d-3) measuring time-density curves of the mask images.

11. The analysis method as claimed in claim 10, wherein in the step (d-2), differentiating the plurality of blood vessel images is obtained by a thresholding method.

12. An analysis system of digital subtraction angiographic images, receiving a time-series dataset of a subject using at least one rotating x-ray source and detector pair, comprising:
(a) an data storage unit, used to storage the time-series dataset of the subject;
(b) an image preprocessing unit, used to perform a motion artifact correction on the time-series dataset;
(c) an image segmentation unit, used to segment the time-series dataset into a plurality of blood vessel images;
(d) a mask generation unit, used to differentiate the plurality of blood vessel images to form a plurality of mask images;
(e) a data processing unit, used to identify time-density curves of the plurality of mask images; and
(f) a medical image interface, used to display the time-density curves, the mask images and the blood vessel images.

13. The analysis system as claimed in claim 12, wherein the time-series dataset are two-dimensional images acquired at an anterior position of the blood vessel, a posterior position of blood vessel, or a lateral positions of the blood vessel.

14. The analysis system as claimed in claim 12, wherein the image preprocessing unit uses a scale-invariant feature transform process to perform the motion artifact correction.

15. The analysis system as claimed in claim 12, wherein the image segmentation unit segments the time-series dataset by using a segmentation method includes a clustering technique, a blind source separation technique, or a machine learning technique.

16. The analysis system as claimed in claim 12, wherein the image segmentation unit processes the time-series dataset by using an independent component analysis method.

17. The analysis system as claimed in claim 12, wherein the time-series dataset segmented by the image segmentation unit is an original time-series dataset, a subtracted time-series dataset, or a calculated parametric dataset.

18. The analysis system as claimed in claim 12, wherein the data processing unit identifies vascular structures at arterial, capillary, and venous phases, and the corresponding time-density curve of these three vascular structures.

19. The analysis system as claimed in claim 17, wherein the subtracted time-series dataset are the images acquired with the contrast media flowing in the blood vessels subtracted by the images acquired before the arrival of the contrast media, and a stationary structure is cancelled out; wherein the stationary structure is bone, gray matter or white matter.

20. The analysis system as claimed in claim 17, wherein the calculated parametric dataset includes time-to-peak images, maximum-enhancement images, or area-under-curve images.

21. The analysis system as claimed in claim 12, wherein the mask generation unit uses a thresholding method to differentiate the plurality of blood vessel images to form a plurality of mask images.
